(19) United States
(12) Patent Application Publication
(10) Pub. No.: US 2006/0229715 A1
(43) Pub. Date: Oct. 12, 2006

(54) Implants incorporating nanotubes and methods for producing the same

(75) Inventors: Naim Istephanous, Roseville, MN (US); Jeffrey P. Rouleau, Maple Grove, MN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/092,603

(22) Filed: Mar. 29, 2005

(51) Int. Cl. A61F 2/28

(52) U.S. Cl. 623/1.46; 623/23.5; 623/23.55; 205/322

(57) ABSTRACT

A surface-modified implant comprising a plurality of nanotubes and a process for preparing the surface-modified implant. The metal-containing surface of the implant is modified using an electrochemical anodization process to create a plurality of nanotubes formed of an oxide of the metal on at least a surface of the implant.

### FIELD OF THE INVENTION

Embodiments of the invention relate to surface modified implants. More particularly, the embodiments relate to implants with nanotube surface modifications formed from the oxide of the metal on the implant surface by an electrochemical anodization process.

### BACKGROUND

Medical implants play an important role in modern medicine. Bone implants or osteoimplants, for example, are used to augment or even replace entire bone structures. For example, metallic osteoimplants are commonly used to replace the femoral head and hip socket of patients requiring hip replacement surgery. Other bone implants include fixation and attachment devices such as screws, plates, and rods. The use of osteoimplants covers a broad spectrum of medicine, including orthodontics, repair of fractured bones, and vertebral disorders. Medical implants such as stents are used to open closed arteries or other ducts in the body. Drug depot implants are used to deliver prolonged releases of incorporated biological agents.

Ideally, an implant has minimal adverse effects on the body (i.e. the implant is biologically inert). For implants in contact with bone, stable fixation is critical for a favorable pain-free clinical result. Various mechanical means including screws, spikes, and keels have been used to create a stable bone-implant interface.

However, direct and intimate attachment of bone to the implant through bone ongrowth or ingrowth may provide the best clinical outcome. Osteointegration refers to the propensity of a medical implant to integrate with adjacent bony structures in a compatible manner. Osteointegration is a function of, inter alia, an implant's osteoconductive and osteoinductive properties. In an effort to increase an implant's osteoconductive and osteoinductive properties, it has been known to apply various coatings to an implant's surface. These coatings range from exotic metallic alloys to porous ceramics and biologically advantageous polymers. Exemplary coatings include hydroxyapatite and tricalcium phosphate, and porous or textured metallic coatings such as plasma sprayed titanium and sintered beaded coatings. Similar strategies have been used to enhance the compatibility and therapeutic effects of other types of medical implants.

While the prior implant coatings have, to varying degrees, improved osteointegration and other advantageous properties of medical implants, there is still room for improvement in this area, as well as in other areas.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

What is needed is an inexpensive, simple method of modifying the surface of an implant in order to impart advantageous osteoconductive and osteoinductive properties to the implant. There also is a need to provide a surface modification to any implant regardless of its geometry, whereby the modification is capable of withstanding deformation during high temperature processing. There also is a need to provide a surface modified implant with improved osteointegration properties that do not deteriorate over time, and that are not damaged during use or implantation. Embodiments of the invention solve some or all of these needs, as well as additional needs.

Therefore, in accordance with an embodiment of the present invention, there is provided an implant having a metal-containing surface, wherein at least the surface comprises nanotubes of an oxide of the metal.

In accordance with another embodiment of the present invention, there is provided a process for modifying the metal-containing surface of an implant wherein metallic oxide nanotubes are formed on the surface of the implant, the method comprising immersing the implant in an acidic electrolyte solution, and applying a voltage between the implant and a cathode to form metallic oxide nanotubes on the surface of the implant.

In accordance with another embodiment, there is provided a process for modifying the surface of an implant having a metal-containing surface comprising: providing an implant having a titanium or titanium alloy surface; immersing the implant and a cathode in an acidic electrolyte solution including hydrofluoric acid; and applying an electrical potential between the implant and the cathode; wherein titanium-containing nanotubes are formed on the surface of the implant.

These and other features and advantages of the present invention will be apparent from the description provide herein.

### BRIEF DESCRIPTION OF THE DRAWINGS

FIG. 1 is a drawing of an exemplary surface modified titanium or titanium alloy-containing implant.

FIG. 2 is a schematic illustrating a mechanism of nanotube formation on a surface of an implant.

FIG. 3 is a schematic illustrating an alternative mechanism of nanotube formation on a surface of an implant.

FIG. 4 is a schematic illustrating an annealing process applied to a surface modified titanium or titanium alloy-containing implant.

FIG. 5 is an image from a field emission scanning electron microscope (FE-SEM) of a surface modified Ti-6Al-4V pedicle screw.

FIG. 6 is an image from a field emission scanning electron microscope (FE-SEM) of a surface modified Ti-6Al-4V pedicle screw after implantation into a porcine vertebrae.

### DETAILED DESCRIPTION OF THE EMBODIMENTS

The following description is intended to convey a thorough understanding of the various embodiments of the invention by providing a number of specific embodiments and details involving surface modified implants incorporating nanotube surface features, preferably surface modified implants intended to be implanted at or near bone, that have enhanced osteointegration due in part to surface modifications including nanotubes. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

Throughout this description, the term “layer” denotes any arrangement whereby one portion of the material has a different chemical composition than another. The layer may include any number of individual layers, and the interface between the layer(s) may be sharp or gradual. For example, a nanotube oxide layer on the surface of an implant having a metal-containing surface may denote a layer deposited on the surface, or may denote a layer formed below the surface by oxidation of the metal on the surface of the implant. The interface between the oxide layer and the underlying implant may be sharp or gradual. For example, the interface between the oxide layer and the underlying implant may comprise a gradual rise in the amount of oxide present from, say 0% in the underlying implant to 50% in the oxide layer, over a certain thickness of the implant.

It is a feature of an embodiment of the present invention to provide a surface modified implant incorporating nanotube surface features. The implants of the embodiments may be of any particular size, form, configuration, or shape. For example, the surface modified implants may be bone screws such as pedicle screws and fixation screws; cylinder implants; blade implants; mandibular implants; hip screws; shaped bone prosthetics; plates; rods; hip, knee, and shoulder replacement parts; fusion cages; and all other types of implants for use at or near bone.

In another embodiment, the implant may be a stent including, but not limited to, arterial, esophagal, biliary, colon, urethral, airway, and lacrimal stents. The stent, for example, may be a balloon expandable stent, self expandable stent, tubular stent, or coil stent. Generally, any stent comprising a substrate or surface of any appropriate metal or metal alloy may be manufactured or modified according to embodiments of the present invention.

In still another embodiment, the implant may be an implantable drug depot used to deliver biological agents such as pharmaceuticals inside the body. Embodiments of the present invention enable the manufacture of drug depots comprising a substrate or surface of any appropriate metal or metal alloy having nanotube surface features.

The implants may comprise a substrate or surface of any appropriate metal or metal alloy, such as titanium, titanium alloys, tantalum, tantalum alloys, stainless steel alloys, cobalt-based alloys, cobalt-chromium alloys, cobalt-chromium-molybdenum alloys, niobium alloys, and zirconium alloys. The substrate may be a surface layer of the metal or metal alloy, or the entire implant may be comprised of the metal or metal alloy. In addition, the expression “metal-containing surface,” as it is used herein, includes the entire surface of the implant containing a metal, or only a portion of the surface containing a metal. In a preferred embodiment, the implant is a bone implant comprising a surface of titanium, a titanium alloy such as Ti-6Al-4V, or tantalum. The metal substrate of the bone implant optionally may be coupled with ceramic and/or plastic structures.

The implant may be subjected to an electrochemical anodization process to modify the surface of the implant. In general, the metal surface of the implant functions as the anode during the electrochemical anodization process. Oxidation of the metal surface of the implant (i.e., the anode surface) occurs and, given appropriate reaction conditions, nanotubes are formed on the surface of the implant.

The surface of the implant may be prepared before the electrochemical anodization process is performed. For example, the implant surface may be cleaned using distilled water and isopropyl alcohol or methyl ethyl ketone washes, optionally combined with ultrasonic agitation of the washes to further help remove impurities from the surface of the implant. The implant surface also may be cleaned by a chemical-mechanical polishing process or simply a mechanical polishing process, for example, using a diamond paste. One who is skilled in the art will appreciate other applicable methods by which the implant surface may be cleaned before performing the anodizing process.

The electrochemical anodization process may occur in a suitable electrolyte solution. Generally, the electrolyte solution is a suitable acidic solution, for example, a chromic acid or sulfuric acid solution. The addition of chromic acid, it is believed, yields an electrolyte solution with Cr₂O₇²⁻ as the predominant species. The concentration of chromic acid in the electrolyte solution preferably may be from about 0.1 to about 1.5 mole per liter of water (mol/L), and more preferably from about 0.25 to about 1 mole per liter of water (mol/L), and most preferably about 0.5 mole per liter of water (mol/L). It is contemplated that other electrolyte solutions also may be successfully used in the electrochemical anodization process.

In the case of titanium and titanium alloy-containing implants, the electrolyte solution additionally may comprise hydrofluoric acid, yielding an electrolyte solution with Cr₂O₇²⁻ and HF as the predominant species. The concentration of HF in the electrolyte solution may preferably be from about 0.1% to about 5% by volume, and more preferably from about 0.3% to about 2.5% by volume, and most preferably from about 0.5% to about 1.5% by volume. It may be preferable to stir the electrolyte solution, for example by magnetic stirring, during the electrochemical anodization process in order to reduce the variation in local temperature and voltage on the surface of the bone implant in the electrolyte solution. Reduction in local variation of temperature and voltage in turn may yield an implant with a more uniform distribution of nanotubes on its surface.

Generally, the specific electrolyte solution used will depend upon the composition of the metallic surface of the implant. Therefore, an electrolyte solution useful for the formation of nanotubes on the surface of a titanium-containing implant may be different, for example, from an electrolyte solution useful for the formation of nanotubes on the surface of a tantalum-containing implant. One skilled in the art will appreciate other electrolyte solutions that successfully may be used in the anodization process to create nanotubes on the surface of the implant.

It may be preferable to choose as a cathode, an inert, corrosion resistant metal. For example, gold, iridium, platinum, rhodium, palladium, and ruthenium are among the metals contemplated for use as the cathode in the electrochemical anodization process. One skilled in the art will appreciate other materials that successfully may be used as the cathode in the electrochemical anodization process.

Electrical potential may be applied between the anode and the cathode placed in the electrolyte solution by an outside electrical source. The electrical potential may vary from about 1 V to about 40 V, preferably from about 5 V to about 35 V, and most preferably from about 10 V to about 30 V. In a preferred embodiment where the implant has a titanium or titanium alloy-containing surface and the concentration of hydrofluoric acid in the electrolyte solution is about 0.5% by volume, the electrical potential may be anywhere from about 10 V to about 30 V. Without desiring to be limited thereto, it is believed that electrical potentials below about 10 V may yield a nanoporous structure on the surface of the titanium or titanium alloy-containing implant, rather than the desired well-defined nanotube structures. Also, electrical potentials above about 30 V may modify the surface of the titanium or titanium alloy-containing implant to form a random sponge-like structure, rather than the desired well-defined nanotube structures. An electrical potential of about 20 V in 0.5% by volume hydrofluoric acid solution is most preferred when the implant surface to be modified comprises titanium or a titanium alloy.

The electrical potential for modification of the surface of the implant may be dependant upon the concentration of the acid in the electrolyte solution. Generally, higher voltages may be needed to produce the desired nanotube surface structures when more dilute acidic electrolyte solutions are used. In the case of titanium and titanium alloy-containing implants, the electrical potential for modification of the surface of the implant also is dependant upon the concentration of hydrofluoric acid in the electrolyte solution. Generally, higher voltages are needed to produce the desired nanotube surface structures in more dilute hydrofluoric acid solutions.

Additionally, the electrical potential may affect physical properties of the nanotubes formed on or in the surface of the implant. In general, higher voltage potentials may yield nanotubes with larger pore diameters. Therefore, by choosing the appropriate voltage, nanotubes with a desired pore diameter may be formed on or in the surface of the implant. Also, the electrical potential may be varied during the electrochemical anodization process, resulting in the formation of nanotubes with a tapered structure. A tapered nanotube structure with a large base and narrow top may be desirable, for example, in order to create reservoirs to trap biological agents and additives on the modified surface of the implants, particularly in the case of drug depots.

The nanotubes created by the electrochemical anodization process are typically oxides of the metallic material present on the surface of the implant. For example, in the case of an implant having a titanium-containing surface, titanium oxide (TiO₂) nanotubes are formed on the surface. In the case of an implant having a Ti-6Al-4V titanium alloy-containing surface, the nanotubes may comprise titanium oxide (TiO₂), aluminum oxide (Al₂O₃), and vanadium oxide (VaO₂). In the case of an implant having an aluminum-containing surface, aluminum oxide (Al₂O₃) nanotubes may be formed. Generally, the oxide nanotubes also may incorporate elements from the electrolyte solution in which the electrochemical anodization process takes place. For example, nanotubes formed on the surface of titanium and titanium alloy-containing implants may incorporate small amounts of fluorine in their structure because hydrofluoric acid may be used in the electrolyte solution for electrochemical anodization of titanium and titanium alloy-containing implants.

It may be advantageous to mix certain additives into the electrolyte solution in anticipation of the additives being incorporated into the nanotubes formed on the surface of the implants. For example, ionic substances may be mixed into the electrolyte solution so that the ionic substances will be incorporated into the nanotubes formed on the surface of the implants. An ionic component in the oxide nanotubes may be advantageous in order to increase the nanotubes' ability to retain beneficial biological agents and additives that are to be adsorbed onto or incorporated into the surface of the implant before, during, or after implantation.

The electrochemical mechanism by which nanotube formation proceeds may vary by material. For example, the electrochemical mechanism by which tantalum-containing implants are modified to form nanotube surfaces may be different than the mechanism by which titanium and titanium alloy-containing implants are modified to form nanotube surfaces. Without desiring to be limited to any theory or mode of operation, it has been proposed that the nanotubes grow on an implant having a surface containing titanium or titanium alloy because of a growth-dissolution mechanism regulated by a competitive poisoning-antidote, as shown in FIG. 2.

In the preferred embodiment shown in FIG. 2, an electrolyte solution of chromic acid and hydrofluoric acid is used to treat a titanium or titanium alloy-containing implant 11. Preferably, the outer titanium or titanium alloy-containing surface of the implant 11 is oxidized to form an oxide layer 10. Chromate ions (Cr⁶⁺) provided by the chromic acid in the electrolyte solution may play a poisoning role, causing the formation of the oxide layer to quickly stop. However, the fluoride ions (F) provided by the hydrofluoric acid may play an antidote role, leading to continued growth of the layer. This growth-dissolution mechanism is evidenced by the observation that spherical particles 12 are believed to form on the titanium surface at the initial stage of the electrochemical anodization process. As the growth-dissolution mechanism continues, a porous structure 14 is formed on the surface of the implant. Under certain conditions, for example the proper electrical potential and concentration of hydrofluoric acid, the porous structure may eventually become a layer of nanotubes on the surface of the implant 11, which are comprised of oxides of the implant's metal-containing surface.

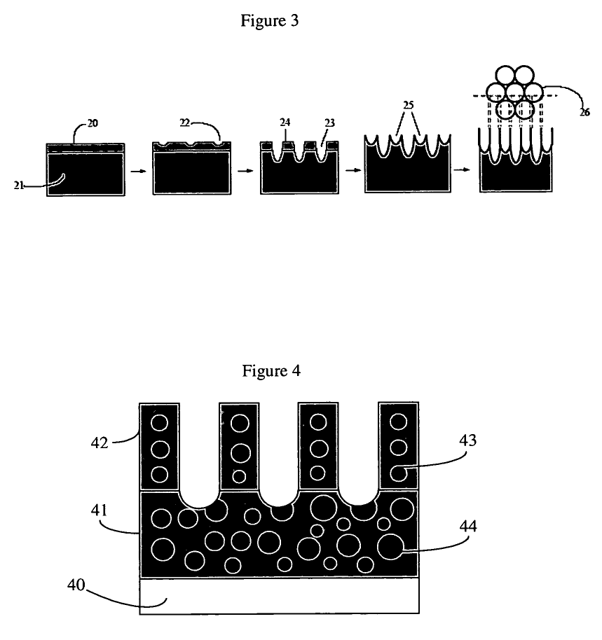

FIG. 3 illustrates another possible mechanism for growth of the nanotube surface layer on titanium or titanium alloy-containing implants. As shown in FIG. 3, a thin oxide layer 20 initially may form on the surface of the implant 21, preferably by oxidation of the outermost metal-containing surface of implant 21. The hydrofluoric acid in the electrolyte solution may cause local dissolution of the oxide layer, forming nano-scale pits 22 in the oxide layer. The pits may increase the electric-field density in the remaining portion of oxide layer 22, causing further pore growth by the deepening and widening of the pores 23. Between the pores exist protrusions of metal and metal oxide 24 and, as the pits deepen, the electric field in the metal protrusions may increase, causing oxide growth and dissolution and the formation of interpore voids 25. The process may continue as the pores and voids grow deeper until the nanotube structure 26 is formed. The view shown above the right-most diagram in FIG. 3 is a top plan view showing the regular array of nanotubes formed from the surface of the implant 21.

An additional mechanism for growth of the nanotube surface on implants having titanium and/or titanium alloy-containing surfaces may involve two processes: (i) field-enhanced oxide dissolution; and (ii) field-enhanced oxidation of titanium. Inside the pore channels there may exist two interfaces: (a) a solution/oxide interface; and (b) an oxide/metal interface. At the oxide/metal interface, electrical field-enhanced oxidation of the metal to form the oxide may occur. At the same time, the electric field may cause titanium ions to migrate from the oxide to the solution/oxide interface and dissolve into solution. In this way, the field-enhanced oxidation of titanium, which converts titanium into titanium oxide, and the field-enhanced oxide dissolution, which subsequently removes titanium oxide from the surface, may cause the oxide layer to grow continuously. Because the electric field may be more intense at the bottom than at the top of the pore, titanium will be consumed at a higher rate near the bottom of the pore, causing the pore to deepen. Eventually, an equilibrium may be established wherein the field-enhanced oxide dissolution and field-enhanced oxidation that is driving the deepening of the pore is equal at the bottom and top of the pores, resulting in a constant pore depth. Additionally, the electric field may cause the inter-pore titanium ions to migrate through the oxide/metal and oxide/solution interfaces into the solution, leaving voids in between the pores and resulting in the creation of the nanotubular structure.

Regardless of the particular mechanism by which the nanotube formation occurs, it is believed that the process of nanotube formation in titanium and titanium alloy-containing implants is such that the layer of nanotubes on the surface of the implant reach a constant depth after which further anodization does not alter the depth of the nanotube layer. In other words, it is believed that the electrochemical anodization process is limited in that it may produce nanotube layers only up to a maximum depth in titanium and titanium alloy-containing implants. Such a limitation may not exist in the surface modification of implants having at least a surface comprised of other metals and alloys.

By adjusting process variables, particularly the voltage, time, and composition of the electrolyte solution, the properties of the nanotubes produced by modifying the surface of the implant may be varied. For example, adjusting the amount of time during which the electrochemical anodization process is executed may affect the depth and formation of the nanotubes. In the formation of nanotubes from titanium and titanium alloy containing surfaces, it has been observed that, within about 5 to 10 seconds of anodization, a compact oxide film may form. After about 30 seconds of anodization, pits begin to form in the oxide film. After about 60 to about 90 seconds of anodization, the pits may become larger pores and spread across the surface of the oxide. After about 120 seconds of anodization, a connected porous structure may be observable with the formation of small pits in the interpore region. After about 8 minutes of anodization, the original oxide film may be completely transformed into a distinct structure of nanotubes. After about 20 minutes of anodization, the nanotube structure may obtain a constant depth. While the time at which these transformations occur may vary dependant upon other processing variables such as voltage and electrolyte composition, it is to be noted that adjusting the process time may be useful to select between different stages in the development of the nanotubes and dimensional characteristics of the nanotubes themselves.

The size of the nanotubes is one property of the nanotubes that may be adjusted by varying process variables such as voltage, time, and composition of the electrolyte solution. It is believed that the electrochemical anodization of a titanium or titanium alloy-containing implant may yield nanotubes with an inner diameter between about 15 nanometers and about 100 nanometers, an outer pore diameter between about 15 nanometers and about 200 nanometers, and a height between about 15 nanometers and about 500 nanometers. However, it also is contemplated that optimization of the electrochemical anodization process as applied to titanium and titanium alloy-containing implants may yield nanotubes with dimensions outside of the these ranges. Additionally, it is contemplated that nanotubes formed from implants comprising other metals and alloys may be produced in different ranges of sizes, dependant upon the metal or alloy that comprises at least the surface of the implant.

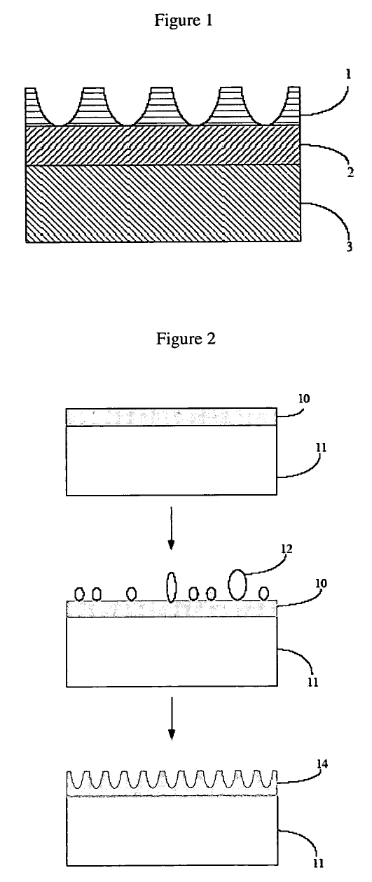

It has been observed, in relation to titanium and titanium alloy-containing implants, that the proper execution of the electrochemical anodization process to form oxide nanotubes on the surface of the implant may result in a three-part structure. On the immediate surface of the implant are the oxide nanotubes, aligned generally perpendicular to the surface geometry of the implant. Below the oxide nanotubes is the interface between the nanotube layer and the titanium surface. The interface may also comprise an oxide of the titanium or titanium alloy. Below the interface between the nanotube layer and the titanium surface is the titanium surface itself. These three layers are depicted in FIG. 1, where 1 indicates the oxide nanotubes, 2 indicates the interface between the oxide nanotubes and the titanium surface, and 3 indicates the titanium surface. Without desiring to be limited to any theory of operation, it is believed that similar structures may be observed in modified surface implants comprising other metals and metal alloy surfaces.

Following electrochemical anodization and formation of nanotubes on the surface, the implant may undergo further treatment to impart advantageous properties to the implant. For example, the surface-modified implants may be annealed to toughen the surface of the implant and to modify the crystalline structure of the nanotubes. For example, the titanium oxide nanotubes formed on the surface of titanium-containing implants are thought to be amorphous in nature. Proper annealing may form either of two crystalline structures that usually are found in titanium oxide crystals—the rutile and anatase crystalline phases. Both the rutile and anatase phases have a similar tetragonal symmetry comprising six Ti—O bonds. However, the rutile phase has a structure based on octagons of titanium dioxide which each share two edges with adjacent octagons, forming chains. In the anatase phase, the structure is based on octagons of titanium dioxide which each share four edges with adjacent octagons. The electrical properties of amorphous, anatase phase, and rutile phase titanium oxide are different and therefore may initiate different biological responses. The annealing process preferably may be executed so as to select between the rutile and anatase phases of titanium oxide in accordance with a desired biological response.

FIG. 4 depicts an exemplary annealing process of titanium bone implants. Without desiring to be limited to any theory or mode of operation, it is thought that, at temperatures of about 230° C. to 280° C. in an oxygen atmosphere, the oxide nanotubes 42 on the surface of a titanium-containing implant and the interface layer of oxide 41 between the titanium surface 40 and the nanotube structures 42 may begin to crystallize to the anatase phase. In other words, anatase phase crystals in the nanotube structures 43 and anatase phase crystals in the interface layer of oxide 44 may begin to form. The anatase phase crystals may grow in size with increased temperatures. At about 430° C., the anatase phase crystals in the interface layer of oxide 44 may transform into the rutile phase, and with increasing temperature also will grow in size. The anatase phase crystals in the nanotube structures 43 typically do not transform into the rutile phase until they have grown large enough to intersect the growing rutile phase crystals in the interface layer of oxide.

One possible mechanism to explain the anatase to rutile phase transformation is that, with rising temperature, the oxygen ion framework of the anatase phase is spatially disturbed and a majority of the Ti⁴⁺ ions are shifted by breaking two of the six Ti—O bonds to form new bonds. It has been proposed that nucleation and growth of the rutile crystals may occur at the interface of two contacting anatase crystals. Additionally, it has been proposed that nucleation and growth of the rutile crystals may occur at the surface or in the bulk of anatase crystals. Also, titanium may be directly oxidized to the rutile phase at sufficiently high temperatures. Rutile nucleation in the walls of the nanotubes does not occur, it is believed, because there is not sufficient space in the nanotube walls for the anatase crystals to rotate and reorient into the rutile phase.

Again without desiring to be limited to any theory or mode of operation, it is thought that the nanotubes of the surface-modified titanium-containing implant usually are stable up to temperatures of about 580° C. in oxygen atmospheres. In dry argon environments, a small amount of pore shrinkage or thinning of the nanotube walls may occur during annealing of the surface-modified implants up to about 580° C. However, if the surface-modified titanium-containing implant is annealed in humid argon environments up to about 580° C., tube shrinkage may be more pronounced. At temperatures exceeding 580° C., the titanium support beneath the nanotube layer on the surface of the implant may begin to oxidize due to the temperature-controlled diffusion of oxygen through the interface layer of oxide to the titanium support. Subsequent crystal growth at the titanium support may destroy the nanotubes on the surface of the implant. Therefore, it may be preferable to anneal the surface-modified titanium-containing implant at temperatures not exceeding 580° C.

Processing variables, for example the time, voltage, temperature, and composition of the electrolyte preferably may be adjusted in order to control, for example, the pore diameter, sidewall thickness, shape, height, and composition of the nanotubes formed on the surface of the implant. One who is skilled in the art will appreciate still other processing variables that may be advantageously adjusted in order to control the modification of the implant and formation of nanotubes thereon in accordance with the embodiments described herein.

For example, it is thought that higher voltage potentials may yield nanotubes with larger pore diameters. Therefore, by choosing an appropriate voltage, nanotubes with a desired pore diameter may be formed. In order to vary the shape of the nanotubes, for example, the electrical potential may be varied during the electrochemical anodization process. This may result in the formation of tapered nanotubes or otherwise irregularly shaped nanotubes. The height and pore diameter of the nanotubes also may be influenced by the composition of the electrolyte solution. For example, a more dilute electrolyte composition may delay nanotube formation, thereby decreasing the height of the nanotubes produced over a given time period compared with a more concentrated electrolyte solution. The composition of the electrolyte also may affect the composition of the nanotubes as it is known that at least some trace amounts of components of the electrolytes may be incorporated into the nanotubes during formation. Also, the duration of time during which the implants are modified may be adjusted to attain desired nanotube structures. For example, increasing the duration of the modification process may result in the creation of nanotubes of increased height and more developed structure.

One possible advantage of the surface-modified implants is that the nanotubes, because they are formed from the same material as the surface of the implant, are more mechanically stable than traditional coating layers, or nanotube-grown layers using materials other than the material found at the surface of the implant. It generally is known to apply coating layers of different materials to the surface of implants to impart osteoinductive, osteoconductive, and other beneficial qualities. Because the coating layers are formed from different materials than the surface of the implant, however, they may have a different elastic modulus. As the implant is subjected to stresses inside the body, or stresses created during implantation, the coating layers may delaminate from the surface of the implant because of the difference in elastic modulus between the coating layer and the surface of the implant. Nanotubes formed of the same material as the surface of the implant, however, are believed to have an elastic modulus more closely approximating the elastic modulus of the implant surface. Therefore, the possibility of damage to the surface of the implant due to stresses inside the body may be reduced.

Another possible advantage of the surface-modified implants is that the creation of nanotubes increases the surface area of the implant. Increased surface area may lead to better mechanical fixation because, in general, the ability to interact with adjacent tissues increases with increased surface area of the implant. Still another possible advantage of the surface-modified implants is that the small dimensions of the nanotube surface features encourages interaction with cells, particularly osteoblasts. Without intending to be limited to any theory of operation, it is thought that small dimensions on the surface of implants mimics the surface features of proteins, for example proteins found on the surface of cells. The mimicking of protein surface features in turn promotes interactions with cells, for example osteoblasts.

Still another possible advantage of the surface-modified implants is the retention of the dimensional requirements of the implants during processing. Whereas some coating process may adversely affect the dimensions of the implant, for example due to the high temperatures required during the coating process, the embodiments described herein provide a low-temperature process that may not significantly affect the dimensions of the implant. This may allow surface-modified implants to be fabricated with more precise and standard dimensions.

Another possible advantage of the surface-modified implants is that the electrochemical anodization process may be successfully utilized even for implants with complex geometries, such as fusion cages, which typically have a hollow, cylindrical configuration with voids in the walls of the cylinder to promote bony ingrowth, and stents, which are generally cylindrical or coil shaped. Some other coating technologies, for example sputtering, may be limited to line-of-sight geometries and therefore are of limited utility for modifying the surface of an implant having a complex geometry.

Yet another possible advantage of the surface-modified implants is that the nanotubes may be used as reservoirs for advantageous biological agents and additives to impart, for example, additional osteoinductive and osteoconductive properties to the surface-modified implants. This may be particularly useful for implants of the present invention that are bone implants, drug eluting stents, or drug depots. In a preferred embodiment, one or more biological agents or additives may be added to the implant before implantation. The biological agents and additives may be adsorbed onto and incorporated into the modified surface comprising nanotubes, by dipping the implant into a solution or dispersion containing the agents and/or additives, or by other means recognized by those skilled in the art. In a more preferred embodiment, the nanotubes will release the adsorbed biological agents and additives in a time-controlled fashion. In this way, the therapeutic advantages imparted by the addition of biological agents and additives may be continued for an extended period of time. It may be desirable to include certain additives in the electrolyte solution used during the electrochemical anodization process in order to increase the adsorptive properties of the nanotubes formed on the surface-modified implant. For example, the inclusion of salts in the electrolyte solution used during the electrochemical anodization process may result in the incorporation of ionic substances into the nanotubes formed on the surface-modified implant. The inclusion of ionic substances in the nanotubes may impart greater adsorptive properties to the nanotubes due to the polar interactions between the nanotubes containing ionic substances and the biological agents and additives.

The biological agents or additives may be in a purified form, partially purified form, recombinant form, or any other form appropriate for inclusion in the surface-modified implant. It is preferred that the agents or additives be free of impurities and contaminants.

For example, growth factors may be included in the surface-modified implant to encourage bone or tissue growth. Non-limiting examples of growth factors that may be included are platelet derived growth factor (PDGF), transforming growth factor b (TGF-b), insulin-related growth factor-I (IGF-I), insulin-related growth factor-II (IGF-II), fibroblast growth factor (FGF), beta-2-microglobulin (BDGF II), and bone morphogenetic factors. Bone morphogenetic factors are growth factors whose activity is specific to bone tissue including, but not limited to, proteins of demineralized bone, demineralized bone matrix (DBM), and in particular bone protein (BP) or bone morphogenetic protein (BMP). Osteoinductive factors such as fibronectin (FN), osteonectin (ON), endothelial cell growth factor (ECGF), cementum attachment extracts (CAE), ketanserin, human growth hormone (HGH), animal growth hormones, epidermal growth factor (EGF), interleukin-1 (IL-1), human alpha thrombin, transforming growth factor (TGF-beta), insulin-like growth factor (IGF-1), platelet derived growth factors (PDGF), and fibroblast growth factors (FGF, bFGF, etc.) also may be included in the surface-modified implant.

Still other examples of biological agents and additives that may be added to the surface-modified implant are biocidal/biostatic sugars such as dextran and glucose; peptides; nucleic acid and amino acid sequences such as leptin antagonists, leptin receptor antagonists, and antisense leptin nucleic acids; vitamins; inorganic elements; co-factors for protein synthesis; hormones; endocrine tissue or tissue fragments; synthesizers; enzymes such as collagenase, peptidases, and oxidases; polymer cell scaffolds with parenchymal cells; angiogenic agents; antigenic agents; cytoskeletal agents; cartilage fragments; living cells such as chondrocytes, bone marrow cells, mesenchymal stem cells, natural extracts, genetically engineered living cells, or otherwise modified living cells; autogenous tissues such as blood, serum, soft tissue, and bone marrow; bioadhesives; periodontal ligament chemotactic factor (PDLGF); somatotropin; bone digestors; antitumor agents and chemotherapeutics such as cis-platinum, ifosfamide, methotrexate, and doxorubicin hydrochloride; immuno-suppressants; permeation enhancers such as fatty acid esters including laureate, myristate, and stearate monoesters of polyethylene glycol; bisphosphonates such as alendronate, clodronate, etidronate, ibandronate, (3-amino-1-hydroxypropylidene)-1,1-bisphosphonate (APD), dichloromethylene bisphosphonate, aminobisphosphonatezolendronate, and pamidronate; pain killers and anti-inflammatories such as non-steroidal anti-inflammatory drugs (NSAID) like ketorolac tromethamine, lidocaine hydrochloride, bipivacaine hydrochloride, and ibuprofen; antibiotics and antiretroviral drugs such as tetracycline, vancomycin, cephalosporin, erythromycin, bacitracin, neomycin, penicillin, polymycin B, biomycin, chloromycetin, streptomycin, cefazolin, ampicillin, azactam, tobramycin, clindamycin, gentamicin, and aminoglycocides such as tobramycin and gentamicin; and salts such as strontium salt, fluoride salt, magnesium salt, and sodium salt.

One skilled in the art will appreciate still other advantageous biological agents or additives that may be added to the surface modified bone implants.

Another potential advantage of the embodiments described herein is the ease with which nanotube structures may be formed on a metal-containing surface of an implant. As described above, the electrochemical anodization process by which the nanotubes are formed is relatively simple, fast, and inexpensive to execute.

In an exemplary embodiment of the invention, a fusion cage having at least a metal surface may be processed as described herein. That is, the fusion cage may be immersed in an appropriate electrolyte solution while an electrical potential is applied between the fusion cage and an appropriate cathode. The process may result in the formation of nanotubes on the metal surfaces of the implant. Because the process is not a line-of-sight process, nanotubes may be formed over all the metal surfaces of the implant, even surfaces not amendable to coating using line-of-sight (e.g. sputtering) coating techniques, such as interior surfaces and structures of the fusion cage. This may be advantageous to induce better osteointegration of the fusion cage with adjacent bony structures.

In another exemplary embodiment of the invention, an implant with a metal substrate may be coated with another metal which is subsequently processed to form a layer of nanotubes thereon. For example, a platinum implant may be coated or only a portion of its surface coated with titanium and then the titanium coating may be processed according to the process described herein in order to form nanotubes on the surface of the titanium coating. The titanium coating, for example, may be formed by sputtering or electroplating a titanium layer on the substrate of the implant. The platinum body of the implant may advantageously function as the cathode during the nanotube formation process. In this fashion, an implant comprising different metals may be fashioned and nanotubes formed on only a portion of the implant.

In another exemplary embodiment of the invention, an implant comprising at least a metal surface may be processed in such a manner as to produce a functionally graded surface structure. For example, an implant may be partially immersed in the electrolyte solution during processing so that only a portion of the metal surface of the implant is processed to form nanotubes thereon. Alternatively, the implant may be gradually immersed or withdrawn from the electrolyte solution during processing so that more developed or taller nanotubes are formed on a portion of the implant's metal surface. An exemplary process for the production of a functionally graded bone screw according to this embodiment would be to immerse only a portion of the bone screw in the electrolyte solution so that nanotubes are formed on only a portion of the bone screw. Alternatively, the bone screw may be gradually immersed or gradually removed from the electrolyte during processing so that a more gradually graded nanotube surface is formed. For example, if the screw is immersed or removed from the electrolyte in a length-wise fashion, a graded nanotube surface spanning the portion of the length of the screw contacted by the electrolyte solution may be formed.

In another exemplary embodiment of the invention, an implant comprising at least a metal surface may be a metal clad implant. For example, the implant may have a cladded metal surface of titanium or titanium alloy combined with another appropriate material.

One who is skilled in the art will appreciate the wide variety of implant configurations that advantageously may be modified in accordance with embodiments of the invention.

The invention now will be described in more detail with reference to the following non-limiting examples.

### EXAMPLES

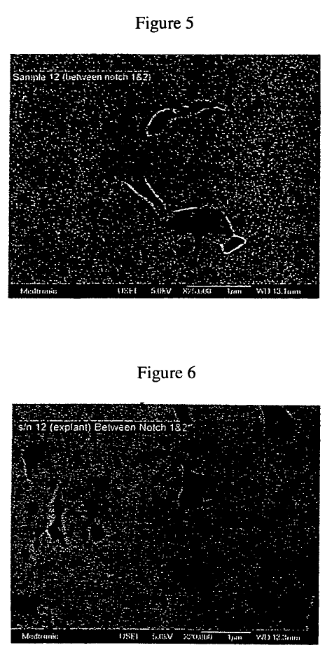

In order to test the stability upon implantation of nanotubes on the surface of an implant, Ti-6Al-4V pedicle screws were processed to form nanotubes on the threads. The pedicle screws were immersed in a 0.5% by weight hydrofluoric acid in water solution. A 20 volt potential was applied to the pedicle screws for 20 minutes at room temperature. The surface-modified pedicle screws were imaged on a field emission scanning electron microscope (FE-SEM—see FIG. 5) prior to insertion into harvested lumbar vertebrae from an adult pig. As can be seen, the surface of the pedicle screw was modified by the anodization process to form a substantially regular array of nanotubes. The screws also were heat treated at 300° C. for two hours prior to implantation.

Lumbar vertebrae from a sacrificed adult male pig were harvested after termination of the animal. Standard procedures of drilling and tapping were used to implant the pedicle screws in the lumbar vertebrae. Following insertion, the surface-modified pedicle screws were carefully removed in a non-contacting fashion using saws and rongeurs rather than by reversing the torque to the screws in order to minimize damage to the nanotubes due to explantation. This was done because only the stability upon implantation of the nanotubes was to be examined; the stability of the nanotubes upon explantation of the implant is largely irrelevant. The surface-modified pedicle screws were again imaged on a FE-SEM (FIG. 6). As shown in FIG. 6, the nanotubes remained in substantially their previous form after the screws where inserted into dense porcine bone. Therefore, it is concluded that the nanotubes formed in accordance with the guidelines provided herein, despite their small dimensions and intricate nature, are sufficiently strong to withstand the stress of implantation into bone.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A surface modified implant having at least a metal-containing surface, comprising a plurality of nanotubes on the surface, where the nanotubes are comprised of an oxide of the metal-containing surface.

2. The surface modified implant of claim 1, where the metal-containing surface comprises a metal selected from the group consisting of titanium, titanium alloys, tantalum, tantalum alloys, stainless steel alloys, cobalt-based alloys, cobalt-chromium alloys, cobalt-chromium-molybdenum alloys, niobium alloys, and zirconium alloys.

3. The surface modified implant of claim 1, where the nanotubes comprise oxides of a metal or alloy selected from the group consisting of titanium, titanium alloys, tantalum, tantalum alloys, stainless steel alloys, cobalt-based alloys, cobalt-chromium alloys, cobalt-chromium-molybdenum alloys, niobium alloys, and zirconium alloys.

4. The surface modified implant of claim 1, where the metal-containing surface comprises commercially pure titanium and the nanotubes comprise titanium oxide.

5. The surface modified implant of claim 1, where the metal-containing surface comprises a titanium alloy and the nanotubes comprise titanium oxide.

6. The surface modified implant of claim 5, where the titanium alloy is Ti-6Al-4V and the nanotubes comprises titanium oxide, aluminum oxide, vanadium oxide, or mixtures and combinations thereof.

7. The surface modified implant of claim 1, where the inner pore diameter of the nanotubes is between about 15 nanometers and about 100 nanometers.

8. The surface modified implant of claim 1, where the outer pore diameter of the nanotubes is between about 15 nanometers and about 200 nanometers.

9. The surface modified implant of claim 1, where the height of the nanotubes is between about 15 nanometers and about 5000 nanometers.

10. The surface modified implant of claim 1, where the nanotubes are formed on the metal-containing surface by an electrochemical anodization process.

11. The surface modified implant of claim 1, where the surface modified implant is an implant selected from the group consisting of a bone implant, stent, drug depot, and fusion cage.

12. A process for modifying a metal-containing surface of an implant to form a plurality of oxide nanotubes on the surface of the implant, comprising:
providing an electrolyte solution;
providing a cathode;
immersing the implant in the electrolyte solution; and
applying a voltage between the implant and the cathode in the electrolyte solution.

13. The process of claim 12, where the metal-containing surface of the implant comprises titanium or a titanium alloy.

14. The process of claim 12, where the oxide nanotubes comprise titanium oxide or oxides of a titanium alloy.

15. The process of claim 12, where the electrolyte solution comprises chromic acid and hydrofluoric acid.

16. The process of claim 12, where the voltage between the implant and the cathode is between about 1 V and about 40 V.

17. The process of claim 12, where a biological agent or additive is adsorbed onto or incorporated into the modified surface of the implant.

18. The process of claim 12, where the implant is an implant selected from the group consisting of a bone implant, stent, drug depot, and fusion cage.

19. A process for modifying the surface of an implant, comprising:
providing an implant comprising at least a surface containing titanium or titanium alloy;
immersing the implant and a cathode in an acidic electrolyte solution including hydrofluoric acid; and
applying an electrical potential between the implant and the cathode;
wherein a plurality of nanotubes of titanium oxide or oxides of the titanium alloy are formed on the surface of the implant.

20. The process of claim 19, where the concentration of hydrofluoric acid in the electrolyte solution is between about 0.5% by weight and about 1.5% by weight.

21. The process of claim 19, where the electrolyte solution comprises a solution of chromic acid and hydrofluoric acid.

22. The process of claim 21, where the concentration of chrornic acid in the electrolyte solution is about 0.5 moles per liter of water.

23. The process of claim 19, where the electrical potential is between about 1 V and about 40 V.

24. The process of claim 23, where the electrical potential is about 20 V.

25. The process of claim 19, where the implant is cleaned before immersing the implant in the electrolyte solution.

26. The process of claim 19, where the implant is annealed after formation of the nanotubes on the surface of the implant.

27. The process of claim 26, where the implant is annealed at a temperature less than about 580° C.

28. The process of claim 27, where the implant is annealed at a temperature between about 280° C. and about 430° C.

29. The process of claim 27, where the implant is annealed at a temperature between about 430° C. and about 580° C.

30. The process of claim 19, where a biological agent or additive is adsorbed onto or incorporated into the modified surface of the implant.

31. The process of claim 30, where the biological agent or additive is selected from the group consisting of sugars; nucleic acid and amino acid sequences; vitamins; inorganic elements; co-factors for protein synthesis; hormones; endocrine tissue or tissue fragments; synthesizers; enzymes; polymer cell scaffolds; angiogenic agents; antigenic agents; cytoskeletal agents; cartilage fragments; living cells; autogenous tissues; somatotropin; bone digesters; antitumor agents and chemotherapeutics; permeation enhancers; bisphosphonates; pain killers and anti-inflammatories; antibiotics and antiretroviral drugs; and salts.

32. The process of claim 19, where the implant is an implant selected from the group consisting of a bone implant, stent, drug depot, and fusion cage.

33. The surface modified implant of claim 1, where the implant additionally comprises an implant body to which the metal-containing surface is attached.

34. The surface modified implant of claim 33, where the metal-containing surface is a metal layer covering all or a portion of the surface of the implant.

35. The surface modified implant of claim 34, where the metal layer is attached to the implant body by cladding the implant body with the metal layer, by depositing the metal layer by sputtering, or by depositing the metal layer by electroplating the metal layer to the implant body.
